(19) United States
(12) Patent Application Publication
(10) Pub. No.: US 2026/0188506 A1
(43) Pub. Date: Jul. 2, 2026

(54) A HEALTH MANAGEMENT SYSTEM

(71) Applicant: Kulbinder SINGH, London (GB)

(72) Inventor: Kulbinder SINGH, London (GB)

(21) Appl. No.: 19/127,771

(22) PCT Filed: Sep. 7, 2023
(86) PCT No.: PCT/IB2023/058868

§ 371 Date: May 7, 2025

(30) Foreign Application Priority Data
Sep. 7, 2022 (GB) 2213034.8

(51) Int. Cl. G16H 50/30; A47G 19/22; A61B 5/00; A61B 5/024; A61B 5/145; G16H 20/60; G16H 70/60

(52) U.S. Cl. CPC G16H 50/30; A47G 19/2227; A61B 5/024; A61B 5/14532; A61B 5/14542; A61B 5/4845; A61B 5/681; A61B 5/742; G16H 20/60; G16H 70/60

(57) ABSTRACT

A system for managing health comprising: a wearable diagnostic device having means for measuring, in use, at least one biometric characteristic of a user, and means for communicating the results of the biometric characteristic measurements; a drinking vessel having means to measure, in use, at least one component of a liquid contained in the drinking vessel, and means for communicating the results of the component measurements; and a computer implemented application which, in use, receives as inputs the results of the biometric characteristic measurements and the results of the component measurements, and communicates outputs, to be presented to the user, to a user interface, the outputs comprising information relating to the management of the user's health, based on the results of the biometric characteristics measurements and the results of the component measurements.

### FIELD OF THE INVENTION

The invention relates to a system for managing health. In particular, the invention relates to a system which analyses a liquid to be ingested, takes biometric signals from a user, and provides health management advice based on the liquid analysis and biometric signals.

### BACKGROUND OF THE INVENTION

Drinking is one of the most fundamental human activities. Thirst is one of the most urgent drives because it directs us to fulfil a most essential need: hydration.

Humans do not only drink to quench their thirst, however. We drink for many reasons, including for social, cultural, religious, medicinal, and nutritional reasons.

The benefits of drinking are many and varied, but the outcomes are not always and inevitably positive. Even drinking water can prove fatal in some cases if too much is consumed in a short time.

Drinking alcoholic drinks in moderation is pleasant for most people but can trigger addiction in others and, according to some studies, even cognitive decline over time. The dangers of drinking alcohol to excess are well documented.

Drinking sugary drinks can be refreshing and energising in appropriate amounts, but can lead to tooth decay, obesity, and diabetes, among other problems.

In addition to the problems that can arise from immoderate use of fundamentally healthful dinks, it is possible to cause deliberate harm by secreting toxic substances in another person's drink, such as with drink ‘spiking’, and even murder by poison.

The nature and quantity of the liquid to be ingested are not the only factors which determine whether the outcome will be positive or negative; the physiology, health status, and other characteristics of the drinker are also relevant. Because there are so many variables, it can be difficult for people to make informed and accurate health decisions about what they drink.

KR 20180130965 discloses a smart drinking cup which identifies the nature of a drink contained therein, including nutritional content, and provides recommendations for user drinking behaviour based on that information.

KR 101925535 discloses a smart cup which determines the psychological state of a user based on immediate biometric readings, such as pulse rate, taken by sensors built into the cup, and recommends drinking behaviour to the user accordingly.

There remains a need for a smart drinking vessel which provides more accurate information and advice related to drinking to a user based on both information relating to the content of a particular drink and the physiological state of the drinker, and this latter not just on immediate short term physiological readings but using ongoing continuous or regular readings of one or more biometric characteristic.

The present invention seeks to contribute to the meeting of this need.

### SUMMARY OF THE INVENTION

The invention provides a system for managing health comprising: a wearable diagnostic device having means for measuring, in use, at least one biometric characteristic of a user, and means for communicating the results of the biometric characteristic measurements; a drinking vessel having means to measure, identify, and/or detect (henceforth, ‘measure’ for brevity), in use, at least one component of a liquid contained in the drinking vessel, and means for communicating the results of the component measurements; and a computer implemented application which, in use, receives as inputs the results of the biometric characteristic measurements and the results of the component measurements, and communicates outputs, to be presented to the user, to a user interface, the outputs comprising information relating to the management of the user's health, based on the results of the biometric characteristics measurements and the results of the component measurements.

The wearable diagnostic device may take a variety of forms. For example, it may take the form of a smart watch equipped with sensors for measuring the pulse rate, perspiration, electrical signals from the heart, and even blood pressure. It may take the form of a transdermal patch from which one or more microneedles protrudes, for extracting bodily fluids and testing them for the presence of substances such as sugar, alcohol, and oxygen. It may comprises a plurality of wearable elements of various types, cooperating to output a more comprehensive set of biometric or diagnostic readings.

The drinking vessel may be any conventional or novel vessel for holding a drinkable liquid and releasing it to the user for drinking. Typical examples include drinking glasses of all shapes and kinds, cups and mugs, flasks, and bottles.

The means for measuring at least one component of a liquid in the drinking vessel may take a variety of forms. For example, a piece of suitably treated filter paper may be secured to the inside of the drinking vessel for chromatographic analysis, in which case a sensor for automatic analysis of the chromatographic results, such as a colour sensor, should also be provided. Alternatively or additionally, a strip treated with a chemical such as glucose oxidase may be inserted into a suitable aperture within the drinking vessel, so that sugar levels or similar within a drink in the vessel can be determined by applying an electrical current across the strip. The skilled person will be aware of the many and various in situ chemical tests which would be suitable for use with the invention.

The wearable diagnostic device and the means for measuring at least one component of a liquid in the drinking vessel are provided with means for communicating the results of their measurements. Preferably, these will be wireless communication means, such as near field communication means, such as Bluetooth. Suitable processing equipment, transceiver equipment and power supply circuitry is provided, but this is not described in detail herein since many suitable existing solutions for this provision will be apparent to the skilled reader.

The user interface may take a variety of forms. For example, it may take the form of an electronic display screen which may optionally be attachable to the drinking vessel of the invention so that the user can clearly see the information presented on it before taking a drink. Such an electronic display screen may have suitable transceiver equipment and processing equipment so as to receive as inputs information from the wearable diagnostic device and from the means for measuring the at least one component of a liquid in the drinking vessel, and determine using the computer implemented application the appropriate information to output to the user based on the inputs. Alternatively, the processor which determines the appropriate outputs may be provided outside the user interface, for example in a cloud computing platform, with the outputs being communicated to the user interface once determined.

In other embodiments, the user interface may be a user's smart device, such as a smart phone or tablet computer, onto which the computer implemented application is installed. In such embodiments, it is preferable that the computer implemented application will instruct the smart device to present push notifications to the user so that the user does not forget to check the health-related information before drinking.

It will be appreciated that the invention has many advantages over the prior art. Because the biometric or diagnostic sensors are provided on a wearable device separate to the drinking vessel, many more types of readings are possible than in prior art drinking vessels which provide touch sensors on, for example, the handle of a cup. It is also possible to take readings continuously, or periodically, using a wearable diagnostic device of the invention, and not only when the user is holding the drinking vessel. This allows more accurate readings to be taken, and also information about trends in the readings, which may be relevant to health management.

Because the means for measuring at least one component of a liquid is provided in a drinking vessel, the user is more likely to make the measurements and receive the information than if a separate test needed to be carried out before pouring a drink. Furthermore, the drinking vessel, being intrinsically portable, can be carried around with the user for use every time s/he takes a drink. For example, it is becoming increasingly common for people to carry a water bottle or a lidded cup with them for frequent re-use.

In some embodiments, one of the biometric characteristics measured by the wearable diagnostic device is blood sugar levels, one of the liquid components measured in the drinking vessel is sugar, and the output information relating to the management of the user's health includes at least one of: information relating to predicted user blood sugar levels after consuming the liquid in the drinking vessel; the impact of consuming the liquid in the drinking vessel on the health of a diabetic user; and/or the effect on a weight loss programme undertaken by the user of consuming the liquid in the drinking vessel.

In some such embodiments, the computer implemented application also receives inputs of information relating to at least one of: the user's weight; the user's body mass index; the user's weight loss goals; the user's metabolic rate; and wherein the output of the computer implemented application is further based on these outputs when they are received.

Controlling one's intake of sugar is desirable for many reasons. For some people, such as those suffering from Diabetes, it is essential to survival. For those seeking to lose weight, it is very useful. Providing a user with a real time indication of the impact of drinking a particular drink will help the user to make an informed decision on how to proceed.

Thus, it would be useful simply to give objective information about the sugar content of the liquid in the drinking vessel, which would give the user information relating to the energy contained in the drink among other things. The invention takes this advantage a further step by combining the objective information relating to the drink with subjective information relating to the user, including personal targets for energy intake, and information relating to metabolism and health conditions. Thus, the invention can inform a user that, for example, drinking a given drink will take them from 85% to 90% of their daily energy intake, or would take their blood sugar to a dangerous level.

In some embodiments, one of the biometric characteristics measured by the wearable diagnostic device is blood alcohol levels, one of the liquid components measured in the drinking vessel is alcohol, and the output information relating to the management of the user's health includes at least one of: information relating to predicted user blood alcohol levels after consuming the liquid in the drinking vessel; and/or the impact of consuming the liquid in the drinking vessel on the user's ability or legal permission to drive.

In some such embodiment, the computer implemented application also receives inputs of information relating to at least one of: the user's weight; the user's body mass index; the user's specified preferred alcoholic inebriation level; and local maximum permitted blood alcohol levels for operating a vehicle; and wherein the output of the computer implemented application is further based on these outputs when they are received.

A user is well served with information already when buying alcoholic drinks, because of various labelling conventions which indicate alcohol content. The user does not, however, always have access to the labels, nor does s/he have any confirmation of their accuracy. For example, when a companion provides a drink to the user, already poured into a glass, there is no indication of alcohol content available. Thus, it will often be useful for the user to be provided with the objective information about alcohol content at the point of drinking an alcoholic drink.

The invention takes this advantage a step further by taking subjective information, such as up-to-the-minute blood alcohol levels and information relating to body mass and metabolic rate, to provide even more useful information. Thus, the invention can inform the user, for example, that consuming a given drink will take his/her blood alcohol levels from x to y, and that it will then be unsafe and/or illegal for him/her to drive for a further z hours.

In some embodiments, one of the biometric characteristics measured by the wearable diagnostic device is one of: heart rate; blood oxygen levels; and muscle oxygen levels; one of the liquid components measured in the drinking vessel is one of: whey protein; casein protein; and other proteins; and the output information relating to the management of the user's health includes at least one of: a recommended intake amount of the liquid; and a recommended time to consume the liquid.

In some such embodiments, the computer implemented application also receives inputs of information relating to at least one of: the user's personal perceived anabolic window; the user's exercise schedule; the user's sleep schedule; and wherein the output of the computer implemented application is further based on these outputs when they are received.

It is common for people seeking to improve their strength or fitness to supplement their intake of protein using protein powders and similar dietary supplements, which are often consumed in the form of a drink. Some research suggests that fitness and strength goals can be achieved more effectively by optimising not only the quantity of supplementary protein consumed, but also the timing. Some researchers believe that there is a period (called the ‘anabolic window’) after a workout during which consuming protein is most effective, and that this period, if it exists, is unique to each individual based on a variety of factors.

The invention can inform the user, for example, that now would be the optimum time to consume half of the protein supplement contained in the drinking vessel, based on identifying a period of intense exercise by measuring, for example, pulse rate and/or perspiration using the wearable diagnostic device.

Although the various means for measuring at least one component of the liquid described so far have relied on chemical analysis of the contents of the drinking vessel, other options are suitable for use with the invention. For example, in addition to or instead of a chemical analysis means in the drinking vessel, the drinking vessel could be provided with a means to identify a substance from its packaging, for example a barcode scanner. Before drinking a drink, the user could scan the drink's packaging using the barcode scanner, and the computer implemented invention could thereby look up the details of the contents of the liquid from an internal or external database of liquid components.

Furthermore, although the various means for measuring at least one component of the liquid described so far have been integral to the drinking vessel, they could alternatively be accessories associated with the drinking vessel. For example, they could be provided within drinking straws or stirring spoons or the like.

In some embodiments, the drinking vessel further comprises means for identifying a dangerous additive added to the liquid contained in the drinking vessel, and wherein the computer implemented invention is configured to alert the user via the user interface in the event that a dangerous additive is detected. The deliberate contamination of another person's drink has a long and unfortunate history, from the infamous poisonings of medieval Europe to the plague of drink spiking in modern bars and clubs. New countermeasures are frequently developed, many of them ingenious. This invention can be used to contribute to the effort against drink contamination; the drinking vessel can be provided with means to detect a contaminant and the user interface can then alert the user before they imbibe.

### BRIEF DESCRIPTION OF DRAWINGS

The invention will be described by means of one or more embodiments, with reference to the following drawings.

FIG. 1 depicts a health management system according to a first embodiment of the invention.

FIG. 2 depicts a health management system according to a second embodiment of the invention.

FIG. 3 depicts a health management system according to a third embodiment of the invention.

### DETAILED DESCRIPTION OF THE DRAWINGS

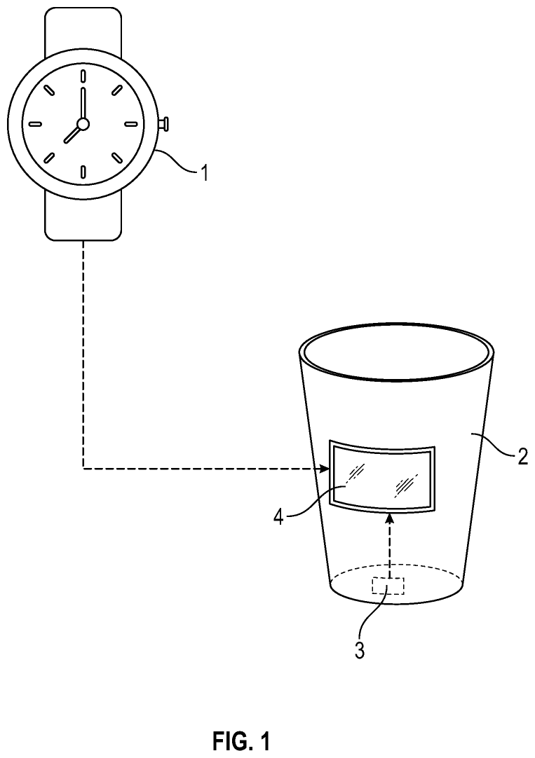

FIG. 1 depicts a first embodiment of the invention, comprising a wearable diagnostic device 1, a drinking vessel 2, a means 3 for measuring at least one component of a liquid in the drinking vessel 2, and a user interface 4. The wearable diagnostic device 1 is depicted in the form of a wristwatch, but the skilled reader will appreciate that it could take many other forms, as discussed above. The wristwatch may include a pulse monitor on its inner face, and a pedometer in its internal workings, as is well known in the art of smart watches.

The wearable diagnostic device is in communication with the user interface 4. This communication is either continuous or periodic. It may be effected, for example, by means of a wireless communications system, such as a near field communication system.

The drinking vessel 2 is depicted in the form of a drinking glass, although the invention can be used with any kind of drinking vessel.

The means 3 for measuring at least one component of a liquid in the drinking vessel is depicted as being embedded in the lower internal surface of the drinking vessel 2, although the skilled reader will appreciate that this is not necessarily the case. The most suitable situation for the means 3 for measuring at least one component of a liquid will depend on the nature of the means 3 and the method by which it makes its measurement.

The means 3 for measuring at least one component of a liquid in the drinking vessel is in communication with the user interface 4. This communication is either continuous or periodic. It may be effected, for example, by means of a wireless communications system, such as a near field communication system.

In the depicted embodiment, both the user interface 4 and the means 3 for measuring at least one component of a liquid in the drinking vessel are attached to the drinking vessel 2, and in some such embodiments the communication between them may be effected by a physical connection, such as by means of optical fibres and/or circuitry.

The user interface 4 in the depicted embodiment takes the form of a small electronic display with a housing which is attached to the drinking vessel 2. It is preferable in such embodiments for the user interface 4 housing to be detachable from the drinking vessel 2 to make it safer and easier to wash the drinking vessel 2 between uses.

In this embodiment, the computer implemented application is stored on memory in the user interface 4 circuitry and implemented by a processor in the user interface 4 circuitry. The user interface 4 circuitry also contains suitable receiver circuitry for receiving inputs from the wearable diagnostic device 1 and the means 3 for measuring at least one component of a liquid.

In some embodiments, it may be desirable for the computer implemented application to send information to either the wearable diagnostic device 1 and/or the means 3 for measuring at least one component of a liquid. For example, the computer implemented application may be configured to instruct one of the other devices to begin operations at a particular time or under particular circumstances, or indeed to stop operations. In such embodiments, the computer implemented application is implemented on a device which controls transmitter circuitry, and the wearable diagnostic device 1 and/or means 3 for measuring at least one component of a liquid have receiver circuitry. In embodiments in which the device which implements the computer implemented application and the means 3 for measuring at least one component of a liquid are physically connected, it may be possible for them to share transmitter and receiver circuitry.

It will be clear to the skilled person that the components of this invention will require power supplies, such as batteries. This is not discussed in detail because it is not necessary for the understanding of the invention.

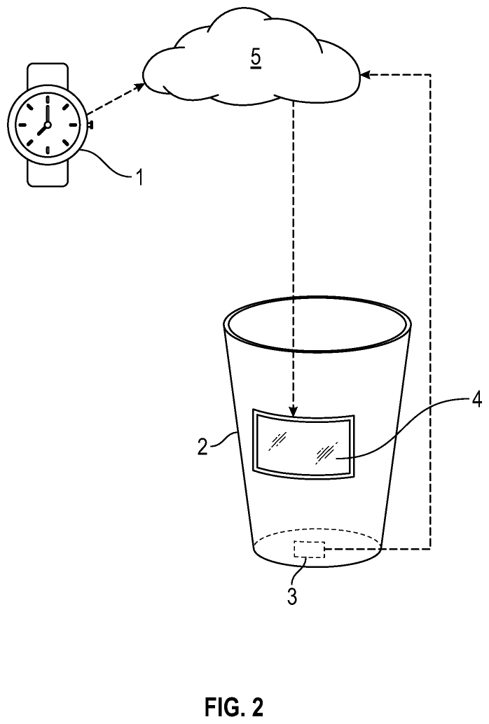

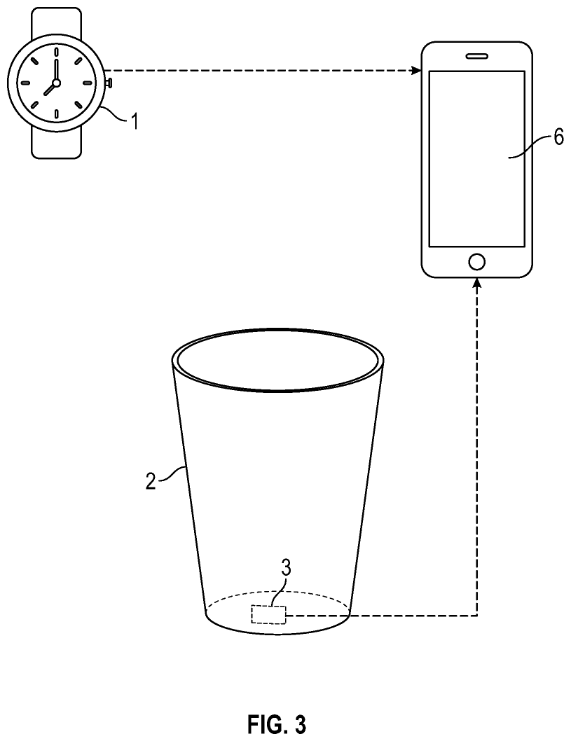

FIG. 2 depicts an embodiment of the invention which is similar to that depicted in FIG. 1, except that the computer implemented application is not implemented by the user interface 4, but rather on a separate computer 5 in remote communication with the other components of the invention. For example, the computer implemented application may be implemented in a cloud computing system 5. In this case, the wearable diagnostic device 1, the means 3 for measuring at least one component of a liquid, and the user interface 4 would each need access to a long distance communications network, such as the Internet, in order to communicate with the remove computer 5. FIG. 3 depicts another embodiment of the invention in which the user interface is in fact a user's smart device, such as a smart phone 6, in which case the computer implemented application can be implemented on the smart phone 6 or on a remote computer in communication with the smart phone 6. Although the invention has been described with reference to a number of preferred embodiments, these embodiments are not limiting. For example, although the discussion of the invention has focussed in particular or measuring sugar, alcohol, and protein components of liquids, there are many uses to which the invention can usefully be put. The invention may be used, for example, to measure the purity of water in the drinking vessel, for example by measuring or detecting one or more of: PH, conductivity, residual chlorine, and/or turbidity.

The scope of the invention is limited only by the claims.

1. A system for managing health comprising:
A wearable diagnostic device having means for measuring, in use, at least one biometric characteristic of a user, and means for communicating the results of the biometric characteristic measurements;
A drinking vessel having means to measure, detect, and/or identify, in use, at least one component of a liquid contained in the drinking vessel, and means for communicating the results of the component measurements, detections, and/or identifications;
And a computer implemented application which, in use, receives as inputs the results of the biometric characteristic measurements and the results of the component measurements, detections and/or identifications, and communicates outputs, to be presented to the user, to a user interface, the outputs comprising information relating to the management of the user's health, based on the results of the biometric characteristics measurements and the results of the component measurements, detections and/or identifications.

2. A system for managing health according to claim 1, wherein one of the biometric characteristics measured by the wearable diagnostic device is blood sugar levels, one of the liquid components measured/identified/detected in the drinking vessel is sugar, and the output information relating to the management of the user's health includes at least one of: information relating to predicted user blood sugar levels after consuming the liquid in the drinking vessel; the impact of consuming the liquid in the drinking vessel on the health of a diabetic user; and/or the effect on a weight loss programme undertaken by the user of consuming the liquid in the drinking vessel.

3. A system for managing health according to claim 2, wherein the computer implemented application also receives inputs of information relating to at least one of: the user's weight; the user's body mass index; the user's weight loss goals; the user's metabolic rate; and wherein the output of the computer implemented application is further based on these outputs when they are received.

4. A system for managing health according to any preceding claim, wherein one of the biometric characteristics measured by the wearable diagnostic device is blood alcohol levels, one of the liquid components measured/identified/detected in the drinking vessel is alcohol, and the output information relating to the management of the user's health includes at least one of: information relating to predicted user blood alcohol levels after consuming the liquid in the drinking vessel; and/or the impact of consuming the liquid in the drinking vessel on the user's ability or legal permission to drive.

5. A system for managing health according to claim 4, wherein the computer implemented application also receives inputs of information relating to at least one of: the user's weight; the user's body mass index; the user's specified preferred alcoholic inebriation level; and local maximum permitted blood alcohol levels for operating a vehicle; and wherein the output of the computer implemented application is further based on these outputs when they are received.

6. A system for managing health according to any preceding claim, wherein one of the biometric characteristics measured by the wearable diagnostic device is one of: heart rate; blood oxygen levels; and muscle oxygen levels; one of the liquid components measured/identified/detected in the drinking vessel is one of: whey protein; casein protein; and other proteins; and the output information relating to the management of the user's health includes at least one of: a recommended intake amount of the liquid; and a recommended time to consume the liquid.

7. A system for managing health according to claim 6, wherein the computer implemented application also receives inputs of information relating to at least one of: the user's personal perceived anabolic window; the user's exercise schedule; the user's sleep schedule; and wherein the output of the computer implemented application is further based on these outputs when they are received.

8. A system for managing health according to any preceding claim wherein the drinking vessel further comprises means for identifying a dangerous additive added to the liquid contained in the drinking vessel, and wherein the computer implemented invention is configured to alert the user via the user interface in the event that a dangerous additive is detected.
